(19) United States
(12) Patent Application Publication
(10) Pub. No.: US 2009/0209030 A1
(43) Pub. Date: Aug. 20, 2009

(54) Thermal Cycler

(76) Inventors: William J. Benett, Livermore, CA (US); James B. Richards, Danville, CA (US); Paul L. Stratton, Brentwood, CA (US); Elizabeth K. Wheeler, Livermore, CA (US); Peter Krulevitch, Pleasanton, CA (US); Steve Visuri, Livermore, CA (US); John M. Dzenitis, Danville, CA (US)

Correspondence Address: Lawrence Livermore National Security, LLC, LAWRENCE LIVERMORE NATIONAL LABORATORY, PO BOX 808, L-703, LIVERMORE, CA, 94551-0808, US

(21) Appl. No.: 12/415,623

(22) Filed: Mar. 31, 2009

Related U.S. Application Data
(63) Continuation-in-part of application No. 10/272,178, filed on Oct. 15, 2002.

(51) Int. Cl. C12M 1/02; H05K 3/00

(52) U.S. Cl. 435/303.1; 29/829

(57) ABSTRACT

A thermalcycler is constructing with a first thermalcycler body section of a flexible circuit material or a circuit board material. The first thermalcycler body section has a first face. A first cavity portion is formed in the first face. A second thermalcycler body section is constructed of a flexible circuit material or a circuit board material. The second thermalcycler body section has a second face. A second cavity portion is formed in the second face. When the first cavity portion and the second cavity portion are positioned together they form a cavity. A thermalcycler unit is positioned in the cavity. The first thermalcycler body section and the second thermalcycler body section are connected together with the first face and the face opposed to each other and the thermalcycler unit operatively connected to the first cavity portion and the second cavity portion.

### CROSS-REFERENCE TO RELATED APPLICATIONS

This application is a Continuation-in-Part of U.S. patent application Ser. No. 10/272,178 filed Oct. 15, 2002 titled, “Thermal Cycler,” which is incorporated herein by this reference.

### STATEMENT AS TO RIGHTS TO INVENTIONS MADE UNDER FEDERALLY SPONSORED RESEARCH AND DEVELOPMENT

The United States Government has rights in this invention pursuant to Contract No. DE-AC52-07NA27344 between the United States Department of Energy and Lawrence Livermore National Security, LLC for the operation of Lawrence Livermore National Laboratory.

### BACKGROUND

1. Field of Endeavor

The present invention relates to thermocyclers and more particularly to a thermalcycler for various operations including polymerase chain reactions, testing for DNA hybridization, isothermal reactions, nucleic acid sequence-based amplification, rolling-circle amplification, incubation for immunoassays, and other uses.

2. State of Technology

U.S. Pat. No. 6,372,486 for a thermo cycler to David M. Fripp issued Apr. 16, 2002 provides the following background information, “Traditionally, scientists have used the technique of the Polymerase Chain Reaction (PCR) to synthesize defined sequences of DNA. This generally involves a three step procedure: separation of the DNA to be amplified (template DNA); annealing of short complimentary DNA sequences (primers) to the template DNA and finally the addition of deoxynucleotides to the primer strands in order to copy the template DNA. This is usually performed in a thermal cycling machine where a cycle of three different temperatures is repeated approximately 25-35 times. Template DNA separation and synthesis steps occur at defined temperatures. However, the temperature at which the primer binds to the DNA, may need optimizing in order for this step to occur efficiently and achieve desirable PCR results. Primer annealing optimization experiments usually involve setting up a number of different experiments where only the primer annealing temperature is varied. The experiment may need to be performed 3 or 4 times in order to determine the optimum binding temperature. These experiments would have to be repeated each time a new set of primers was required for different PCRs. The development of a temperature gradient block enables the scientists to determine the optimum binding temperatures of several primer sets in a single experiment.”

U.S. Patent Application Publication No. 2002/0072112 for a thermal cycler for automatic performance of the polymerase chain reaction with close temperature control to John Atwood published Jun. 13, 2002 provides the following background information, “Applications of PCR technology are now moving from basic research to applications in which large numbers of similar amplifications are routinely run. These areas include diagnostic research, biopharmaceutical development, genetic analysis, and environmental testing. Users in these areas would benefit from a high performance PCR system that would provide the user with high throughput, rapid turn-around time, and reproducible results. Users in these areas must be assured of reproducibility from sample-to-sample, run-to-run, lab-to-lab, and instrument-to-instrument.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a thermalcycler constructing with a first thermalcycler body section of a flexible circuit material or a circuit board material. The first thermalcycler body section has a first face. A first cavity portion is formed in the first face. A second thermalcycler body section is constructed of a flexible circuit material or a circuit board material. The second thermalcycler body section has a second face. A second cavity portion is formed in the second face. When the first cavity portion and the second cavity portion are positioned together they form a cavity. A thermalcycler unit is positioned in the cavity. The first thermalcycler body section and the second thermalcycler body section are connected together with the first face and the face opposed to each other and the thermalcycler unit operatively connected to the first cavity portion and the second cavity portion.

In one embodiment the present invention provides a thermalcycler for processing a sample holder that includes a first thermalcycler body having a first face. A second thermalcycler body includes a second face. A cavity for receiving the sample holder is formed in at least one of the first face or the second face. A thermalcycling unit is operatively connected to the cavity. In one embodiment the first thermalcycler body and the second thermalcycler body include a portion made of a flexible circuit material. In another embodiment the first thermalcycler body and the second thermalcycler body include a portion made of a circuit board material. In another embodiment the first thermalcycler body and the second thermalcycler body include a portion made of an insulated flexible heating material. In another embodiment the cavity is in a shape to receive a tubular flow through sample holder. In another embodiment the cavity is in a shape to receive a batch sample holder. The present invention also provides a method of constructing a thermalcycler. The method includes various steps. A first thermalcycler body section having a first face is constructed. A second thermalcycler body section having a second face is constructed. A cavity is formed in at least one of the first face or the second face. A thermalcycler unit is operatively connected to the cavity. The first thermalcycler body section and the second thermalcycler body section are positioned together wherein the first face and the face are opposed to each other. In one embodiment at least a portion of the first thermalcycler body and the second thermalcycler body are constructed of a flexible circuit material. In another embodiment at least a portion of the first thermalcycler body and the second thermalcycler body are constructed of a circuit board material. In another embodiment at least a portion of the first thermalcycler body and the second thermalcycler body are constructed of an insulated flexible heating material. The method of constructing a thermalcycler includes an embodiment wherein the cavity is formed in a shape to receive a tubular flow through sample holder. In another embodiment the method of constructing a thermalcycler includes an embodiment wherein the cavity is formed in a shape to receive a batch sample holder.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 shows structural details and the operation of an embodiment of a thermal-cycling system.

FIG. 2 shows another embodiment of a thermal-cycling system constructed in accordance with the present invention.

FIG. 3 shows another embodiment of a thermal-cycling system constructed in accordance with the present invention.

FIG. 4 shows another embodiment of a thermal-cycling system constructed in accordance with the present invention.

### DETAILED DESCRIPTION OF THE INVENTION

Referring now to the drawings, to the following detailed information, and to incorporated materials; a detailed description of the invention, including specific embodiments, is presented. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

The present invention provides a thermalcycler for various operations including polymerase chain reaction, testing for DNA hybridization, isothermal reaction, nucleic acid sequence-based amplification, rolling-circle amplification, incubation for immunoassay, and other uses. The present invention provides a thermalcycler for processing a sample holder. A first thermalcycler body includes a first face. A second thermalcycler body includes a second face. A cavity for receiving the sample holder is formed in at least one of the first face or the second face. A thermalcycling unit is operatively connected to the cavity. The present invention also provides a method of constructing a thermalcycler. The method includes various steps. A first thermalcycler body section having a first face is constructed. A second thermalcycler body section having a second face is constructed. A cavity is formed in at least one of the first face or the second face. A thermalcycler unit is operatively connected to the cavity. The first thermalcycler body section and the second thermalcycler body section are positioned together wherein the first face and the second face facing each other.

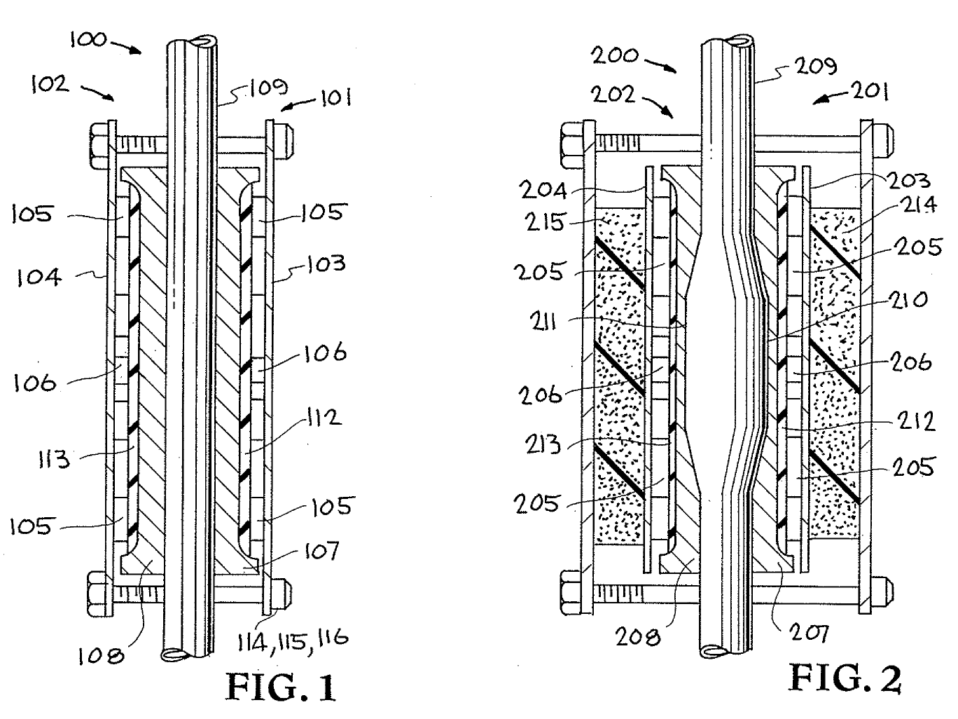

Referring now to FIG. 1, the structural details and operation of an embodiment of a thermalcycling system constructed in accordance with the present invention is illustrated. The thermalcycling system is designated generally by the reference numeral 100. The system 100 also includes a method of constructing a thermalcycler. The method includes various steps. A first thermalcycler body section having a first face and a second thermalcycler body section having a second face. The first thermalcycler body section and the second thermalcycler body section are constructed of a flexible circuit material or a circuit board material. A cavity is formed in at least one of the first face or the second face. A thermalcycler unit is operatively connected to the cavity. The first thermalcycler body section and the second thermalcycler body section are positioned together with the first face and the second face opposed.

There is an increasing need to build smaller more portable thermalcyclers for use in the field and clinical settings. There is a growing need to imbed thermalcyclers in more complex autonomous systems. It is also becoming important to reduce the cost of instruments without sacrificing performance. The thermalcycling system 100 addresses the demonstrated need for a very portable, rapid instrument that can be constructed inexpensively. Uses of the thermalcycling system 100 include pathology, forensics, detection of biological warfare agents, detection of bio-terrorism agents, infectious disease diagnostics, genetic testing, environmental testing, environmental monitoring, point-of care diagnostics, rapid sequencing, detection of biowarfare/bio-terrorism agents in the field, polymerase chain reactions, testing for DNA hybridization, isothermal reactions, nucleic acid sequence-based amplification, rolling-circle amplification, incubation for immunoassays, and other uses.

Description of Specific Embodiment—System 100

The thermalcycling system 100 comprises a first thermalcycler body section 101 and a second thermalcycler body section 102. The thermalcycler body sections include two frame structures 103 and 104. A pad 105 is located on the frame structure section 103. The pad 105 comprises a precision resistor. The resistor 105 provides heating of the unit. Another pad 106 is located on the frame structure section 103. The pad 106 comprises an RTD. The RTD provides temperature sensing of the unit. In one embodiment a fan is provided which aids in rapid thermal cycling. In one embodiment a pneumatic air source is provided which aids in rapid thermal cycling. In one embodiment the thermalcycler 100 includes passages to allow forced air-cooling by fan or pneumatic air source which aids in rapid thermal cycling.

A first chamber unit 107 is positioned between the first thermalcycler body 101 and a tube 109. The tube 109 is a sample holder that can be used for polymerase chain reactions, testing for DNA hybridization, isothermal reactions, nucleic acid sequence-based amplification, rolling-circle amplification, incubation for immunoassays, and other uses. The sample holder tube 109 is used for flow through thermalcycling according to well know techniques. Instead of a sample holder tube for flow through thermalcycling, the sample holder 109 can be a test tube or a sample holder of various shapes.

A second chamber unit 108 is positioned between the second thermalcycler body section 102 and the tube 109. The first and second chamber units 107 and 108 are thermally conductive. In one embodiment the first and second chamber units 107 and 108 are made of copper. Copper provides good thermal conductivity. The first and second chamber units 107 and 108 have cavities 110 and 111 respectively for receiving the sample holder tube 109. The first thermalcycler body section 101, the second thermalcycler body section 102, and frame structure sections 103 and 104 are be made of a flexible circuit material or a circuit board material.

Flexible Circuit Material

The term “flexible circuit material” as used in this patent application means: a porous, expanded polytetrafluoroethylene material. The term “Flexible Circuit Material” used in the specific embodiment system 100 means a porous, expanded polytetrafluoroethylene material impregnated with a bismaleimide-triazine resin. The “Flexible Circuit Material” used in another embodiment includes porous, expanded polytetrafluoroethylene material laminated to a glass cloth. The flexible circuit material will allow the heating resistor section 105 and RTD section 106 to move and align themselves to the surface of the chamber sections 107 and 108.

Circuit Board Material

The term “Circuit Board Material” as used in this patent application means: an insulating fiberglass reinforced epoxy board material. The “Circuit Board Material” used in the specific embodiment system 100 means: an insulating fiberglass reinforced epoxy board material with difunctional epoxy resin. The circuit board material will allow the frame structures to be very thin, thereby allowing the components to be thermally close coupled to the other components and to the sample holder tube 109.

A first thermally conductive pad 112 is positioned between the first frame structure section 103 and the first chamber unit 107. A second thermally conductive pad 113 is positioned between the second frame structure section 104 and the second chamber unit 108. The first and the second conductive pads 112 and 113 consist of a thermally conductive material. In one embodiment the first and second conductive pads 112 and 113 are made of a conductive elastomer material.

The first thermal cycler body section 101 and the second thermalcycler body section 102 have a first face and a second face respectively. The sample holder tube 109 is positioned in the cavity formed in the two cavity sections 110 and 111 in the first and second chamber units 107 and 108. The first and second chamber units 107 and 108 and the first and the second conductive pads 112 and 113 are held in operative position between the frame structure sections 103 and 104. The first thermal cycler body section 101 and the second thermalcycler body section 102 are positioned together with that the first face and the second face facing each other and are connected together. Various means may be used to connect the first thermal cycler body section 101 and the second thermalcycler body section 102 together. As shown in FIG. 1 bolts 115 are positioned in holes 114 and secured by nuts 116 to connect the first thermal cycler body section 101 and the second thermalcycler body section 102 together.

The thermal cycler 100 can be constructed using different construction methods. In one embodiment the copper chamber sections 107 and 108 are fabricated using circuit board etching technology which is inexpensive and lends itself to mass production. In one embodiment the frame structure sections 103 and 104 are be made of a flexible circuit material. In another embodiment the frame structure sections 103 and 104 are made of a circuit board. In other embodiments the frame structures 103 and 104 are larger and multiple thermalcycling units are positioned between the frame structures 103 and 104. In this embodiment additional sample holder tubes 109, precision resistors 105 for heating of the unit, and RTD 106 for temperature sensing of the unit are positioned between the frame structures 103 and 104.

In one embodiment the thermal cycler 100 is constructed using microfabrication technologies. Microfabrication technology enables the production of electrical, mechanical, electromechanical, optical, chemical and thermal devices, including pumps, valves, heaters, mixers, and detectors for microliter to nanoliter quantities of gases, liquids, and solids. Probes and sensors can now be produced on a microscale. The integration of these microfabricated devices into a single system allows for the batch production of reactor-based analytical instruments. Such integrated microinstruments may be applied to biochemical, inorganic, or organic chemical reactions to perform biomedical and environmental diagnostics, as well as biotechnological processing and detection. The microfabrication technologies include sputtering, electrodeposition, low-pressure vapor deposition, photolithography, and etching. Microfabricated devices are usually formed on crystalline substrates, such as silicon and gallium arsenide, but may be formed on non-crystalline materials, such as glass or certain polymers. The shapes of crystalline devices can be precisely controlled since etched surfaces are generally crystal planes, and crystalline materials may be bonded by processes such as fusion at elevated temperatures, anodic bonding, or field-assisted methods. The operation of integrated microinstruments is easily automated, and since the analysis can be performed in situ, contamination is very low. Because of the inherently small sizes of such devices, the heating and cooling can be extremely rapid. These devices have very low power requirement and can be powered by batteries or by electromagnetic, capacitive, inductive or optical coupling. The small volumes and high surface-area to volume ratios of microfabricated reaction instruments provide a high level of control of the parameters of a reaction.

The thermalcycler 100 can be used for pathology, forensics, detection of biological warfare agents, detection of bio-terrorism agents, infectious disease diagnostics, genetic testing, environmental testing, environmental monitoring, point-of care diagnostics, rapid sequencing, detection of biowarfare/bio-terrorism agents in the field, polymerase chain reactions, testing for DNA hybridization, isothermal reactions, nucleic acid sequence-based amplification, rolling-circle amplification, incubation for immunoassays, and other uses. Specific examples include polymerase chain reaction, testing for DNA hybridization, temperature control of isothermal reactions such as Invader (˜63 C), Nucleic Acid Sequence-based Amplification (NASBA) (˜41 C), Rolling-Circle Amplification or Strand-Displacement Amplification (RCA or SDA), and incubation for immunoassays and cells. The sample holder 109 is in intimate contact with the thermal chamber sections 107 and 108. In the case of a shaped sample holder this is accomplished by inflating the tube while heated. In the design with a straight tube 109 the same process is be used or the chamber units 107 and 108 are pressed on to the tube 109. The intimate contact assists with temperature accuracy of the system 100. The system 100 can be made small which provides very rapid heating and cooling due to the low thermal mass. Very small volumes translates into less usage of very expensive biological reagents. In one embodiment the thermally conductive pad is replaced by thermally conductive adhesives or grease for making good thermal contact. In another embodiment, either or both of the sections 101 or 102 has windows that allow optical interrogation and detection of the sample.

The thermalcycler 100 is constructed with a first thermalcycler body section of a flexible circuit material or a circuit board material and a second thermalcycler body section of a flexible circuit material or a circuit board material. The thermalcycler 100 includes a first cavity portion in the first face and a second cavity portion in the second face. When the first face and the second face are positioned opposite each other the first cavity portion and the second cavity portion together form a cavity. A sample holder is positioned in the cavity for receiving the sample. The sample holder is in full direct contact with said the face and the second face. The thermalcycler 100 provides improved and “unexpected” results and benefits. These results and benefits include the fact that the thermalcycler is extremely fast in operation. The thermalcycler 100 has been built and tested and field data shows robustness with over a year of constant service with over 100,000 thermal cycles and the thermalcycler is still functioning well.

Referring now to FIG. 2, the structural details and the operation of another embodiment of a thermalcycling system constructed in accordance with the present invention is illustrated. This embodiment of a thermalcycling system is designated generally by the reference numeral 200. The thermalcycling system 200 comprises a first thermalcycler body section 201 and a second thermalcycler body section 202. The thermalcycler body sections include two frame structures 203 and 204. A cavity, formed by the cavity section 210 and 211, is provided in the thermalcycler body sections 201 and 202. At least one thermalcycling unit is operatively connected to the cavity. It is understood that multiple thermalcycling units can be positioned between the frame structures 203 and 204. Multiple sample holder tubes, precision resistors for heating of the units, and RTDs for temperature sensing of the units can be positioned between the frame structures 203 and 204.

A first chamber unit 207 is positioned between the first thermalcycler body 201 and a tube 209. The tube 209 is a sample holder that can be used for polymerase chain reactions, testing for DNA hybridization, isothermal reactions, nucleic acid sequence-based amplification, rolling-circle amplification, incubation for immunoassays, and other uses. The sample holder can also be a test tube or a sample holder of various shapes. A second chamber unit 208 is positioned between the second thermalcycler body section 202 and the tube 209. The first and second chamber units 207 and 208 are thermally conductive. In one embodiment the first and second chamber units 207 and 208 are made of copper. Copper provides good thermal conductivity. The first and second chamber units 207 and 208 have cavities 210 and 211 respectively for receiving the tube 209.

The first thermalcycler body section 201, the second thermalcycler body section 202, and frame structure sections 203 and 204 are be made of a flexible circuit material or a circuit board material. The terms “flexible circuit material” and “circuit board material” are described above. The flexible circuit material will allow the heating resistor section 205 and RTD section 206 to move and align themselves to the surface of the chamber sections 207 and 208. The circuit board material will allow the frame structures to be very thin, thereby allowing the components to be thermally close coupled to the other components and to the sample holder tube 209.

A component 205 is located on the frame structure section 203. The component 205 comprises a precision resistor. The surface mount resistors and RTDs are mounted or soldered to pads on the circuit board or flexcircuit. The resistor 205 provides heating of the unit. Another component 206 is located on the frame structure section 203. The component 206 comprises an RTD. The RTD provides temperature sensing of the unit.

A first conductive pad 212 is positioned between the first frame structure section 203 and the first chamber unit 207. A second conductive pad 213 is positioned between the second frame structure section 204 and the second chamber unit 208. The first and the second conductive pads 212 and 213 consist of a conductive material. In one embodiment the first and second conductive pads 212 and 213 are made of a conductive elastomer material. A first foam pad 214 is positioned adjacent the first frame structure section 203 on the opposite side of the frame structure 203 from the first conductive pad 212. A second foam pad 215 is positioned adjacent the second frame structure section 204 on the opposite side of the frame structure 204 from the second conductive pad 213.

The first thermal cycler body section 201 and the second thermalcycler body section 202 have a first face and a second face respectively. The first thermal cycler body section 201 and the second thermalcycler body section 202 are positioned together with that the first face and the second face facing each other and are connected together. The first and second chamber units 207 and 208 and the first and the second conductive pads 212 and 213 are held in operative position between the frame structure sections 203 and 204. The first thermal cycler body section 201 and the second thermalcycler body section 202 are connected together. Various means may be used to connect the first thermal cycler body section 201 and the second thermalcycler body section 202 together such as bolts, screws, fasteners, bands, frames, etc. The sample holder unit 209 is positioned in the cavity formed by the two cavity sections 210 and 211 in the first and second chamber units 207 and 208.

The first foam pad 214 and the second foam pad 215 allow the heating resistors 205 and RTD 206 to move and align themselves to the surface of the chamber 207 and 208. The foam elastomer pads 214 and 215 act as a compressive spring to force the heating resistors 205 and RTD 206 into intimate contact with the conductive pads 212 and 213 and chamber 207 and 208. The foam elastomer pads 214 and 215 also act as a layer of thermal insulation.

The system 200 also provides a method of constructing a thermalcycler. The method includes various steps. A first thermalcycler body section 201 having a first face is constructed. A second thermalcycler body section 202 having a second face is constructed. A cavity 210, 211 is formed in at least one of the first face or the second face. A thermalcycler unit is operatively connected to the cavity. The first thermalcycler body section and the second thermalcycler body section are positioned together wherein the first face and the second face facing each other.

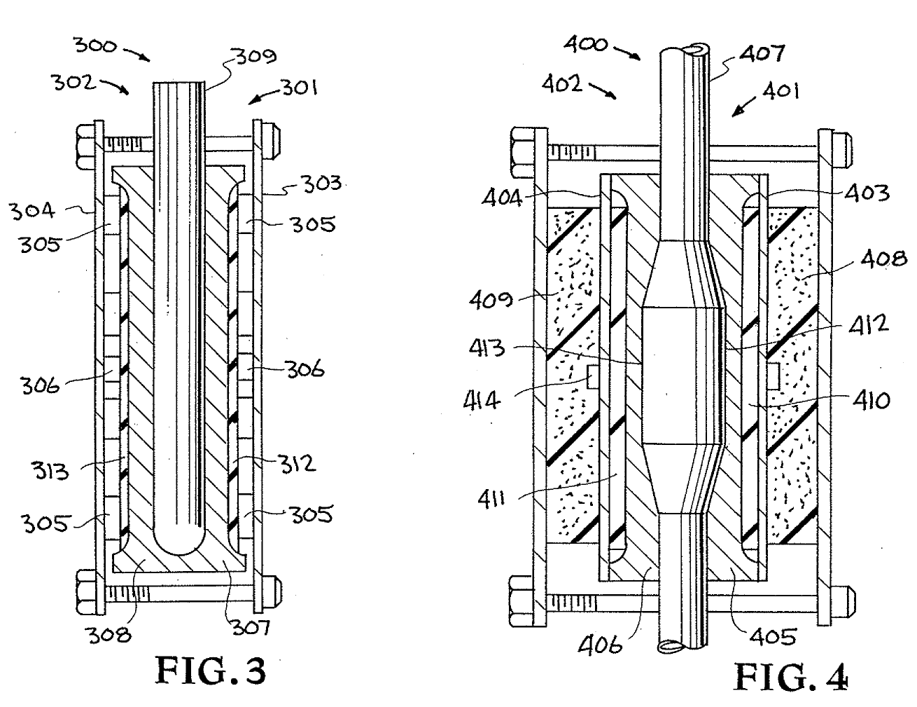

Referring now to FIG. 3, the structural details and the operation of another embodiment of a thermalcycling system constructed in accordance with the present invention is illustrated. The thermalcycling system is designated generally by the reference numeral 300. The thermalcycling system 300 comprises a first thermalcycler body section 301 and a second thermalcycler body section 302. The thermalcycler body sections include two frame structures 303 and 304. A pad 305 is located on the frame structure section 303. The component 305 comprises a precision resistor. The resistor 305 provides heating of the unit. Another pad 306 is located on the frame structure section 303. The component 306 comprises an RTD. The RTD provides temperature sensing of the unit.

A first chamber unit 307 and a second chamber unit 308 are positioned between the two frame structures 303 and 304. A batch sample holder 309 is adapted to be received in a cavity formed by cavity sections 310 and 311 in the chamber units 307 and 308. The sample holder 309 can be a test tube that can be used for polymerase chain reactions, testing for DNA hybridization, isothermal reactions, nucleic acid sequence-based amplification, rolling-circle amplification, incubation for immunoassays, and other uses. The sample holder can also be other forms of batch tubes or a sample holders of various shapes. A second chamber unit 308 is positioned between the second thermalcycler body section 302 and the test tube 309. The first and second chamber units 307 and 308 are thermally conductive. In one embodiment the first and second chamber units 307 and 308 are made of copper. Copper provides good thermal conductivity. The first and second chamber units 307 and 308 have cavities 310 and 311 respectively for receiving the test tube 309.

The first thermalcycler body section 301, the second thermalcycler body section 302, and frame structure sections 303 and 304 are be made of a flexible circuit material or a circuit board material. The terms “flexible circuit material” and “circuit board material” are described above. The flexible circuit material will allow the heating resistor section 305 and RTD section 306 to move and align themselves to the surface of the chamber sections 307 and 308. The circuit board material will allow the frame structures to be very thin, thereby allowing the components to be thermally close coupled to the other components and to the sample holder test tube 309.

A first conductive pad 312 is positioned between the first frame structure section 303 and the first chamber unit 307. A second conductive pad 313 is positioned between the second frame structure section 304 and the second chamber unit 308. The first and the second conductive pads 312 and 313 consist of a thermally conductive material. In one embodiment the first and second conductive pads 312 and 313 are made of a thermally conductive elastomer material.

The first thermal cycler body section 301 and the second thermalcycler body section 302 have a first face and a second face respectively. The first thermal cycler body section 301 and the second thermalcycler body section 302 are positioned together with that the first face and the second face facing each other and are connected together. The first and second chamber units 307 and 308 and the first and the second conductive pads 312 and 313 are held in operative position between the frame structure sections 303 and 304. The sample holder unit 309 is positioned in the cavity formed by the two cavity sections 310 and 311 in the first and second chamber units 307 and 308.

The thermal cycler 300 can be constructed using different construction methods. In one embodiment the copper chamber sections 307 and 308 are fabricated using circuit board etching technology which is inexpensive and lends itself to mass production. The first thermalcycler body section 301, the second thermalcycler body section 302, and frame structure sections 303 and 304 are be made of a flexible circuit material or a circuit board material. The terms “flexible circuit material” and “circuit board material” are described above. In one embodiment the thermal cycler 300 is constructed using microfabrication technologies.

Microfabrication technology enables the production of electrical, mechanical, electromechanical, optical, chemical and thermal devices, including pumps, valves, heaters, mixers, and detectors for microliter to nanoliter quantities of gases, liquids, and solids. Probes and sensors can now be produced on a microscale. The integration of these microfabricated devices into a single system allows for the batch production of reactor-based analytical instruments. Such integrated microinstruments may be applied to biochemical, inorganic, or organic chemical reactions to perform biomedical and environmental diagnostics, as well as biotechnological processing and detection. The microfabrication technologies include sputtering, electrodeposition, low-pressure vapor deposition, photolithography, and etching. Microfabricated devices are usually formed on crystalline substrates, such as silicon and gallium arsenide, but may be formed on non-crystalline materials, such as glass or certain polymers. The shapes of crystalline devices can be precisely controlled since etched surfaces are generally crystal planes, and crystalline materials may be bonded by processes such as fusion at elevated temperatures, anodic bonding, or field-assisted methods. The operation of integrated microinstruments is easily automated, and since the analysis can be performed in situ, contamination is very low. Because of the inherently small sizes of such devices, the heating and cooling can be extremely rapid. These devices have very low power requirement and can be powered by batteries or by electromagnetic, capacitive, inductive or optical coupling. The small volumes and high surface-area to volume ratios of microfabricated reaction instruments provide a high level of control of the parameters of a reaction.

The thermalcycler 300 can be used for pathology, forensics, detection of biological warfare agents, detection of bio-terrorism agents, infectious disease diagnostics, genetic testing, environmental testing, environmental monitoring, point-of care diagnostics, rapid sequencing, detection of biowarfare/bio-terrorism agents in the field, polymerase chain reactions, testing for DNA hybridization, isothermal reactions, nucleic acid sequence-based amplification, rolling-circle amplification, incubation for immunoassays, and other uses. Specific examples include polymerase chain reaction, testing for DNA hybridization, temperature control of isothermal reactions such as Invader (˜63 C), Nucleic Acid Sequence-based Amplification (NASBA) (˜41 C), Rolling-Circle Amplification or Strand-Displacement Amplification (RCA or SDA), and incubation for immunoassays and cells. The sample holder 309 is in intimate contact with the thermal chamber sections 307 and 308. In the case of a shaped sample holder this is accomplished by inflating the test tube while heated. In the design with a straight tube 309 the same process is be used or the chamber units 307 and 308 are pressed on to the tube 309. The intimate contact assists with temperature accuracy of the system 300. The system 300 can be made small which provides very rapid heating and cooling due to the low thermal mass. Very small volumes translates into less usage of very expensive biological reagents. In one embodiment the thermally conductive pad is replaced by thermally conductive adhesives or grease for making good thermal contact. In another embodiment, either or both of the sections 301 or 302 has windows that allow optical interrogation and detection of the sample.

Referring now to FIG. 4, the structural details and the operation of another embodiment of a thermalcycling system constructed in accordance with the present invention is illustrated. This embodiment of a thermalcycling system is designated generally by the reference numeral 400.

The thermalcycling system 400 comprises a first thermalcycler body section 401 and a second thermalcycler body section 402. The thermalcycler body sections include two frame structures 403 and 404. The first thermalcycler body section 401, the second thermalcycler body section 402, and frame structure sections 403 and 404 are be made of a flexible circuit material or a circuit board material. The terms “flexible circuit material” and “circuit board material” are described above. The insulated flexible heating material can be made of silicone rubber, fiberglass, kapton, or other similar materials. Insulated flexible heating material is commercially available. For example, insulated flexible heating material may be obtained from OMEGA Engineering, Inc., One Omega Drive, Stamford, Conn. 06907-0047 or IMI Scott Limited, Dallimore Road, Roundthorn Industrial Estate, Wythenshawe, Manchester M23 9WJ, England.

A first chamber unit 405 and a second chamber unit 406 form part of the thermalcycler body sections 401 and 402. The first and second chamber units 405 and 406 are thermally conductive. In one embodiment the first and second chamber units 405 and 406 are made of copper. Copper provides good thermal conductivity. The first and second chamber units 405 and 406 have cavities 412 and 413 respectively for receiving the sample tube 407. A first conductive pad 41 is positioned between the first frame structure section 403 and the first chamber unit 405. A second conductive pad 413 is positioned between the second frame structure section 404 and the second chamber unit 306. The first and the second conductive pads 412 and 413 consist of a conductive material. In one embodiment the first and second conductive pads 412 and 413 are made of a conductive elastomer material.

A sample holder 407 is positioned in the cavities 412 and 413 in the first chamber unit 405 and a second chamber unit 406 respectively. The sample holder 407 is a sample holder that can be used for polymerase chain reactions, testing for DNA hybridization, isothermal reactions, nucleic acid sequence-based amplification, rolling-circle amplification, incubation for immunoassays, and other uses. The sample holder can be a sample tube for flow through processing, a test tube, or a sample holder of various shapes and designs.

The thermalcycler body sections include two frame structures 403 and 404. The two frame structures 403 and 404 are made of an insulated flexible heating material comprising silicone rubber, fiberglass, kapton, or other similar materials. The insulated flexible heating frame structures 403 and 404 provide reliability, cost effectiveness, minimum cross-section, resistance to deterioration, and basic flexibility. Temperature control is provided by sensor and control element 414 attached to one of the frame structures 403 or 404. The sensor and control unit 414 provides temperature control and sensing by sensing some change in a physical characteristic Six types of sensor and control elements are: thermocouples, resistive temperature devices (RTDs and thermistors), infrared radiators, bimetallic devices, liquid expansion devices, and change-of-state devices. The sensor and control element 414 is commercially available, and for example may be obtained from OMEGA Engineering, Inc., One Omega Drive, Stamford, Conn. 06907-0047 or IMI Scott Limited, Dallimore Road, Roundthorn Industrial Estate, Wythenshawe, Manchester M23 9WJ, England.

The first thermal cycler body section 401 and the second thermalcycler body section 402 have a first face and a second face respectively. The first thermal cycler body section 401 and the second thermalcycler body section 402 are positioned together with that the first face and the second face opposing each other. The first thermal cycler body section 401 and the second thermalcycler body section 402 are connected together. Various means may be used to connect the first thermal cycler body section 401 and the second thermalcycler body section 402 together such as bolts, screws, fasteners, bands, frames, etc, The sample holder unit 407 is positioned in the cavity formed by the two cavity sections 412 and 413 in the first and second chamber units 405 and 406.

The first and second chamber units 405 and 406 are held in operative position between the frame structure sections 403 and 404. A first foam pad 408 is positioned adjacent the first frame structure section 403 on the opposite side of the frame structure 403 from the first chamber unit 405. A second foam pad 415 is positioned adjacent the second frame structure section 404 on the opposite side of the frame structure 404 from the second chamber unit 406. The foam elastomer pads 408 and 409 act as a compressive spring to force the components into contact with the chamber units 405 and 406 and the sample holder 407. The foam elastomer pads 408 and 409 also act as a layer of thermal insulation.

The system 400 also provides a method of constructing a thermalcycler. The method includes various steps. A first thermalcycler body section 401 having a first face is constructed. A second thermalcycler body section 402 having a second face is constructed. A cavity is formed by the cavity sections 412 and 413 in the first and second chamber units 405 and 406. The cavity receives a sample holder 407. The thermalcycler 400 system provides a thermalcycler that can be used for pathology, forensics, detection of biological warfare agents, detection of bio-terrorism agents, infectious disease diagnostics, genetic testing, environmental testing, environmental monitoring, point-of care diagnostics, rapid sequencing, detection of biowarfare/bio-terrorism agents in the field, polymerase chain reactions, testing for DNA hybridization, isothermal reactions, nucleic acid sequence-based amplification, rolling-circle amplification, incubation for immunoassays, and other uses.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A thermalcycler for processing a sample, comprising:
a first thermalcycler body unit having a first face, said first thermalcycler body unit made of a flexible circuit material or a circuit board material;
a second thermalcycler body unit having a second face, said second thermalcycler body unit made of a flexible circuit material or a circuit board material;
a first cavity portion in said first face;
a second cavity portion in said second face;
wherein said first face and said second face are positioned opposite each other and said first cavity portion and said second cavity portion together form a cavity;
a sample holder positioned in said cavity for receiving the sample, wherein said sample holder is in full direct contact with said first face and said second face; and
a thermalcycling heating unit in at least one of said first thermalcycler body unit or said second thermalcycler body unit, said thermalcycling heating unit operatively connected to said sample holder.

2. The thermalcycler of claim 1 wherein said first thermalcycler body unit and said second thermalcycler body unit are made of a flexible circuit material.

3. The thermalcycler of claim 1 wherein said first thermalcycler body unit and said second thermalcycler body unit are made of a circuit board material.

4. The thermalcycler of claim 1 wherein said sample holder is a tubular sample holder and said cavity is in a shape to receive said tubular sample holder.

5. The thermalcycler of claim 1 wherein said sample holder is a tubular flow through sample holder and said cavity is in a shape to receive said tubular flow through sample holder.

6. The thermalcycler of claim 1 wherein said sample holder is a batch sample holder and said cavity is in a shape to receive said batch sample holder.

7. The thermalcycler of claim 1 wherein said thermalcycling heating unit comprises a precision resistor.

8. A thermalcycler for processing a sample, comprising:
a first thermalcycler body unit having a first face, said first thermalcycler body unit made of a flexible circuit material or a circuit board material;
a second thermalcycler body unit having a second face, said second thermalcycler body unit made of a flexible circuit material or a circuit board material;
a first cavity portion in said first face;
a second cavity portion in said second face;
wherein said first face and said second face are positioned opposite each other and said first cavity portion and said second cavity portion together form a cavity;
a sample holder positioned in said cavity for receiving the sample, wherein said sample holder is in full direct contact with said first face and said second face;
a first frame structure positioned adjacent said first thermalcycler body unit;
a second frame structure positioned adjacent said second thermalcycler body unit; and
a thermalcycling heating unit in at least one of said first thermalcycler body unit or said second thermalcycler body unit said thermalcycling heating unit operatively connected to said sample holder.

9. The thermalcycler of claim 8 wherein said first thermalcycler body unit and said second thermalcycler body unit are made of a flexible circuit material.

10. The thermalcycler of claim 8 wherein said first thermalcycler body unit and said second thermalcycler body unit are made of a circuit board material.

11. The thermalcycler of claim 8 wherein said sample holder is a tubular sample holder and said cavity is in a shape to receive said tubular sample holder.

12. The thermalcycler of claim 8 wherein said sample holder is a tubular flow through sample holder and said cavity is in a shape to receive said tubular flow through sample holder.

13. The thermalcycler of claim 8 wherein said sample holder is a batch sample holder and said cavity is in a shape to receive said batch sample holder.

14. The thermalcycler of claim 8 wherein said thermalcycling heating unit comprises a precision resistor.

15. The thermalcycler of claim 8 wherein said first cavity portion in said first face contains a layer of copper and wherein said second cavity portion in said second face contains a layer of copper.

16. The thermalcycler of claim 8 including a first foam pad between said first thermalcycler body unit and said first frame structure and including a second foam pad between said second thermalcycler body unit and second first frame structure.

17. A method of constructing a thermalcycler, comprising the steps of:
constructing a first thermalcycler body section of a flexible circuit material or a circuit board material, said first thermalcycler body section having a first face;
forming a first cavity portion in said first face;
constructing a second thermalcycler body section of a flexible circuit material or a circuit board material, said second thermalcycler body section having a second face;
forming a second cavity portion in said second face, wherein said first cavity portion and said second cavity portion form a cavity;
positioning a thermalcycler unit in said cavity operatively connected to said first cavity portion and said second cavity portion; and
connecting said first thermalcycler body section and said second thermalcycler body section together wherein said first face and said face are opposed to each other and said thermalcycler unit is operatively connected to said first cavity portion and said second cavity portion.

18. The method of constructing a thermalcycler of claim 17, wherein said first thermalcycler body section and said second thermalcycler body section are constructed of a flexible circuit material.

19. The method of constructing a thermalcycler of claim 17, wherein said first thermalcycler body section and said second thermalcycler body section are constructed of a circuit board material.

20. The method of constructing a thermalcycler of claim 17, including providing a heating device in said thermalcycling unit.

21. A method of constructing a thermalcycler, comprising the steps of:
constructing a first thermalcycler body section of a flexible circuit material or a circuit board material, said first thermalcycler body section having a first face;
forming a first cavity portion in said first face;
constructing a second thermalcycler body section of a flexible circuit material or a circuit board material said second thermalcycler body section having a second face;
forming a second cavity portion in said second face, wherein said first cavity portion and said second cavity portion form a cavity;
positioning a thermalcycler unit in said cavity operatively connected to said first cavity portion and said second cavity portion;
connecting said first thermalcycler body section and said second thermalcycler body section together wherein said first face and said face are opposed to each other and said thermalcycler unit is operatively connected to said first cavity portion and said second cavity portion;
positioning a first frame structure adjacent said first thermalcycler body section;
positioning a second frame structure adjacent said second thermalcycler body section; and
connecting said first frame structure and said second frame structure.

22. The method of constructing a thermalcycler of claim 21, wherein said first thermalcycler body section and said second thermalcycler body section are constructed of a flexible circuit material.

23. The method of constructing a thermalcycler of claim 21, wherein said first thermalcycler body section and said second thermalcycler body section are constructed of a circuit board material.

24. The method of constructing a thermalcycler of claim 21 including the steps of providing a layer of copper in said first cavity portion of said first face and providing a layer of copper in said second cavity portion of said second face.

25. The method of constructing a thermalcycler of claim 21, including providing a heating device in said thermalcycling unit.
